(19) United States
(12) Patent Application Publication
(10) Pub. No.: US 2013/0251633 A1
(43) Pub. Date: Sep. 26, 2013

(54) SYSTEMS, METHODS, AND DEVICES FOR ULTRASONIC ASSESSMENT OF CANCER AND RESPONSE TO THERAPY

(71) Applicants: Mark Andrew Borden, Boulder, CO (US); Jessica Kandel, New York, NY (US); Shashank Ramesh Sirsi, New York, NY (US); Darrell Yamashiro, Westwood, NJ (US)

(72) Inventors: Mark Andrew Borden, Boulder, CO (US); Jessica Kandel, New York, NY (US); Shashank Ramesh Sirsi, New York, NY (US); Darrell Yamashiro, Westwood, NJ (US)

(73) Assignee: The Trustees of Columbia University in the City of New York, New York, NY (US)

(21) Appl. No.: 13/813,236

(22) PCT Filed: Aug. 5, 2011
(86) PCT No.: PCT/US11/46830

§ 371 Date: Jun. 14, 2013

Related U.S. Application Data
(60) Provisional application No. 61/371,091, filed on Aug. 5, 2010.

(51) Int. Cl. A61K 49/22

(52) U.S. Cl. CPC A61K 49/223
USPC 424/9.2

(57) ABSTRACT

Microbubbles can be injected into the bloodstream of a patient, for example, a cancer patient undergoing a treatment specifically targeting a biological process in a tumor. The injected microbubbles can act as vascular contrast agents, which can be detected in vivo using high-frequency ultrasound imaging. The microbubbles can have a surface chemistry that allows them to bind to molecular targets in the tumor vasculature. After injection, the microbubbles can selectively adhere to endothelia expressing a target receptor. The selective adhesion can be used to quantify the tumor vasculature in vivo. By imaging the adhered microbubbles with ultrasound, an indication of how tumor vasculature is affected by a specific cancer treatment can be obtained. Such techniques can be used in a clinical setting for rapid determination of anti-cancer treatment efficacy for individual patients.

### CROSS-REFERENCE TO RELATED APPLICATIONS

The present application claims the benefit of U.S. Provisional Application No. 61/371,091, filed Aug. 5, 2010, which is hereby incorporated by reference herein it its entirety.

### STATEMENT REGARDING FEDERALLY SPONSORED RESEARCH

The present invention was made with U.S. Government support under grant/contract no. R21-CA-139173 awarded by National Institutes of Health (NIH)/National Cancer Institute (NCI). The U.S. Government has certain rights in the invention.

### FIELD

The present disclosure relates generally to microbubbles, and, more particularly, to specially formulated microbubbles and methods for the production and use thereof. The present disclosure also relates to cancer assessment techniques, and, more particularly, ultrasonic assessment of tumor response to therapies using specially formulated microbubbles.

### BACKGROUND

Cancer treatments that target specific biological process in tumors (e.g., blood vessel development) can be highly effective for certain subsets of patients. However, not all patients may respond to this type of treatment. For example, vascular endothelial growth factor (VEGF) is a key mediator of tumor angiogenesis. A humanized monoclonal anti-VEGF antibody, bevacizumab (BV), has been developed and validated as a potential cancer therapy, but efficacy may vary depending on cancer type and individual patients. A patient may thus undergo treatment while tumor morphology is monitored in order to ascertain if the treatment is effective. During this time, the tumor may grow and/or the cancer may spread to other parts of the body if the treatment is ineffective. The effectiveness variability among patients for these cancer treatments may exact significant physical and financial tolls, not the least of which is the lost opportunity to switch to a potentially more effective treatment before further cancer progression. While biologically targeted agents may hold promise for increasing effectiveness of cancer treatments, it may be advantageous in clinical practice to determine as early as possible whether a patient would respond to BV or other cancer treatments.

### SUMMARY

Microbubbles can be formulated with a specific surface chemistry in order to bind to molecular targets within a patient, such as, but not limited to, target receptors in a tumor. Microbubbles injected into the bloodstream of the patient can circulate and eventually bind to the target receptors. The bound microbubbles can then be imaged using high-frequency ultrasound to allow in vivo visualization of the vasculature in the region of interest (ROI) of the patient.

In particular, specially formulated microbubbles can be injected into the bloodstream of a patient, for example, a cancer patient undergoing a treatment specifically targeting a biological process in a tumor. The injected microbubbles can act as vascular contrast agents, which can subsequently be detected using high-frequency ultrasound imaging. For example, the microbubbles can have diameters of 4-5 μm and/or 6-8 μm. The microbubbles can have a surface chemistry that allows them to bind to molecular targets in the tumor vasculature. After injection, the microbubbles can selectively adhere to endothelia expressing a target receptor. The selective adhesion can be used to quantify the tumor vasculature in vivo. By imaging the adhered microbubbles with ultrasound, an indication of how tumor vasculature is affected by a specific cancer treatment can be obtained. Such techniques can be used in a clinical setting for rapid determination of anti-cancer treatment efficacy for individual patients.

In embodiments, a method for determining efficacy of treatment of a cancerous tumor in a patient can include, at a first time after administering the treatment to a patient, injecting a population of microbubbles into the patient. The population of microbubbles can be size-selected so as to have diameters within a specified range. Each microbubble can have a surface chemistry that targets receptor sites in the tumor. After the injecting, a field of view can be imaged using ultrasound so as to obtain a first image. The field of view can include at least a portion of the tumor. The method can further include, after the imaging, sending an ultrasonic pulse to the field of view so as to destroy the microbubbles therein. The ultrasonic pulse can have a higher intensity than the ultrasound waves used for the imaging. The field of view can be re-imaged using ultrasound so as to obtain a second image. The method can also include comparing the intensity of the first and second images so as to measure the number of microbubbles attached to the targeted receptor sites in the tumor.

In embodiments, a substance for investigation of the efficacy of an anti-cancer treatment can include a plurality of microbubbles in solution. Each microbubble can have a gas core surrounded by a lipid membrane. The lipid membrane can have a surface chemistry that binds to receptor sites in a cancerous tumor. The respective diameters of the plurality of microbubbles can be 4-5 μm and/or 6-8 μm.

Objects and advantages of embodiments of the disclosed subject matter will become apparent from the following description when considered in conjunction with the accompanying drawings.

### BRIEF DESCRIPTION OF DRAWINGS

Embodiments will hereinafter be described with reference to the accompanying drawings, which have not necessarily been drawn to scale. Where applicable, some features may not be illustrated to assist in the illustration and description of underlying features. Throughout the figures, like reference numerals denote like elements.

FIG. 1A is a schematic diagram of a microbubble, according to one or more embodiments of the disclosed subject matter.

FIG. 1B shows graphs of number percentage and volume percentage of microbubbles in size-selected populations, according to one or more embodiments of the disclosed subject matter.

FIG. 1C is a graph of ultrasound intensity as a function of time for different size-selected microbubble populations, according to one or more embodiments of the disclosed subject matter.

FIGS. 2A-2B are graphs of ultrasound intensity and half-life, respectively, as a function of microbubble concentration for different size-selected microbubble populations, according to one or more embodiments of the disclosed subject matter.

FIG. 3A-3B are graphs of ultrasound intensity and half-life, respectively, as a function of microbubble population gas volume for different size-selected microbubble populations, according to one or more embodiments of the disclosed subject matter.

FIG. 4 is a process flow diagram for ultrasonic assessment of tumor response to therapy, according to one or more embodiments of the disclosed subject matter.

FIGS. 5A-5B are graphs of measured ultrasound intensities of in vivo control microbubbles and RGD-peptide microbubbles, respectively, according to one or more embodiments of the disclosed subject matter.

FIG. 6A is a graph of percent change in relative blood volume after treatment with BV for the control microbubbles, according to one or more embodiments of the disclosed subject matter.

FIG. 6B is a graph of percent change in molecular expression after treatment with BV for the RGD-peptide microbubbles, according to one or more embodiments of the disclosed subject matter.

FIGS. 7A-7B show high frequency ultrasound images following bolus injections of size-selected microbubbles into mice implanted with SK-NEP-1 renal tumors and with NGP renal tumors, respectively, according to one or more embodiments of the disclosed subject matter.

FIGS. 7C-7D are graphs of relative microbubble perfusion in SK-NEP-1 tumor regions and NGP tumor regions, respectively, as a function of time, according to one or more embodiments of the disclosed subject matter.

FIG. 8A shows high frequency ultrasound images at day 0 of treatment of mice implanted with SK-NEP-1 renal tumors before (left) and after (right) ultrasonic burst pulse application with corresponding video intensity-time curves (below), according to one or more embodiments of the disclosed subject matter.

FIG. 8B shows high frequency ultrasound images at day 3 of treatment of mice implanted with SK-NEP-1 renal tumors before (left) and after (right) ultrasonic burst pulse application with corresponding video intensity-time curves (below), according to one or more embodiments of the disclosed subject matter.

FIG. 8C shows high frequency ultrasound images at day 0 of treatment of mice implanted with NGP renal tumors before (left) and after (right) ultrasonic burst pulse application with corresponding video intensity-time curves (below), according to one or more embodiments of the disclosed subject matter.

FIG. 8D shows high frequency ultrasound images at day 3 of treatment of mice implanted with NGP renal tumors before (left) and after (right) ultrasonic burst pulse application with corresponding video intensity-time curves (below), according to one or more embodiments of the disclosed subject matter.

FIGS. 8E-8F are graphs of relative targeted microbubble adhesion in SK-NEP-1 tumor regions and NGP tumor regions, respectively, as a function of time, according to one or more embodiments of the disclosed subject matter.

FIG. 9A-9B are fluorescent images of SK-NEP-1 tumors injected with fluorescein-labeled lectin at day 0 and day 3, respectively, after the start of treatment, according to one or more embodiments of the disclosed subject matter.

FIG. 9C-9D are fluorescent images of NGP tumors injected with fluorescein-labeled lectin at day 0 and day 3, respectively, after the start of treatment, according to one or more embodiments of the disclosed subject matter.

FIGS. 10A-10B are graphs of estimated microvessel density of SK-NEP-1 tumor vasculatures and NGP tumor vasculatures, respectively, as a function of time, according to one or more embodiments of the disclosed subject matter.

FIGS. 10C-10D are graphs of estimated total vessel length of SK-NEP-1 tumor vasculatures and NGP tumor vasculatures, respectively, as a function of time, according to one or more embodiments of the disclosed subject matter.

FIG. 11 is a graph of two-dimensional cross-sectional area of a tumor as a function of time, according to one or more embodiments of the disclosed subject matter.

FIG. 12A is a graph of initial relative microbubble perfusion in SK-NEP-1 and NGP tumors, according to one or more embodiments of the disclosed subject matter.

FIG. 12B is a graph of initial relative targeted microbubble adhesion in SK-NEP-1 and NGP tumors, according to one or more embodiments of the disclosed subject matter.

### DETAILED DESCRIPTION

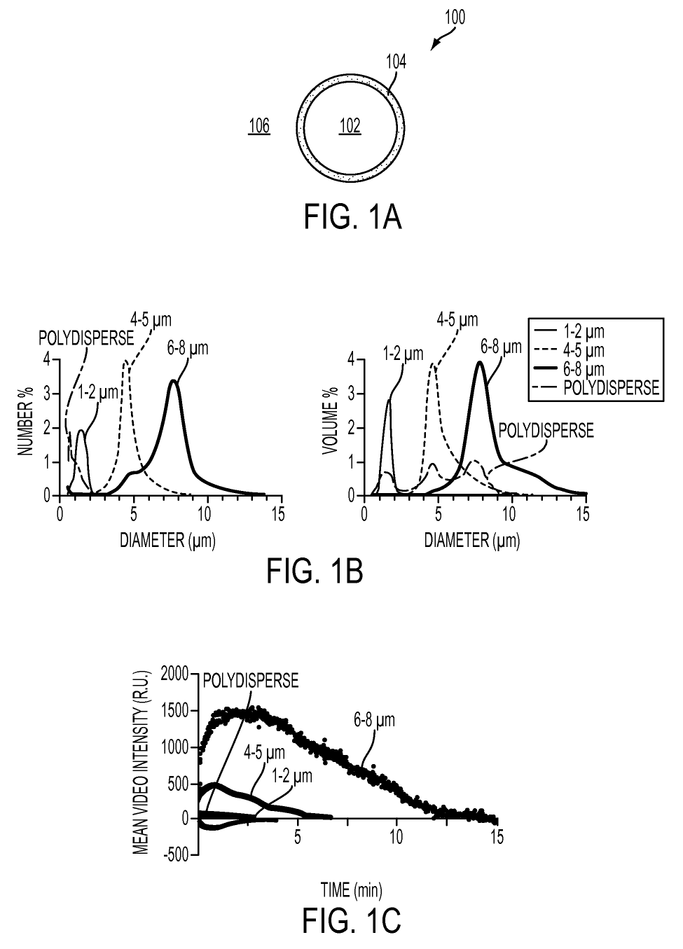

As shown in FIG. 1A, a microbubble 100 is a gas-filled sphere ranging in diameter from approximately 1 μm up to 10 μm. The microbubble 100 can include a shell 104, which can be a lipid, protein, polymer, or combination thereof. Shell 104 separates a gas 102 contained in the interior of the shell 104 from a liquid environment 106. Such a liquid environment can be a liquid solution used to form the microbubble 100, a solution used to store or hold the microbubble 100, or a biological fluid, such as the bloodstream of a patient.

When injected into the bloodstream, microbubbles can act as vascular contrast agents that are detectable using high-frequency ultrasound imaging. The unique ability to distinguish the presence of the microbubbles in circulation from endogenous blood and tissue allow them to be used as probes for mapping vasculatures, for example, tumor vasculatures. Furthermore, microbubbles can be modified to contain ligands on their surface so as to bind to molecular targets in the vasculature. By using an appropriate ligand, the microbubbles, acting as ultrasound imaging contrast agents, can be selectively adhered to endothelia expressing a target receptor in order to quantify the vasculature in vivo. When applied to tumor vasculature, it can indicate how a particular cancer treatment affects tumor vasculature and thus provide a measure of the efficacy of treatment on a patient's tumor.

Ultimately, such techniques could be used in a clinical setting for rapid determination of anti-cancer treatment efficacy with respect to cancerous tumors in individual patients. Moreover, the ability to monitor changes in tumor vasculature during treatment can provide a more expedient determination of anti-cancer therapy efficacy. By providing a determination of treatment therapy efficacy early in the treatment plan, cancer patients can switch to alternative and potentially more effective treatments without wasting precious time on ineffective modalities. Additionally or alternatively, such techniques could be used to monitor vasculature changes in other bodily organs and/or structures beside cancerous tumors.

For example, cyclic arginine-glycine-aspartic acid (RGD) peptides is a ligand that binds to αᵥβ₃ integrin receptors, which are up-regulated in angiogenic blood vessels. Microbubbles can be modified to contain these RGD peptides on their surface so as to function as probes for identifying areas of high αᵥβ₃ integrin expression. The utilization of RGD-targeted and untargeted microbubbles can be used to study the changes in tumor vessel architecture and molecular expression on the surface of blood vessels, for example, after the administration of a therapeutic anti-cancer treatment.

The methodology described herein can be applied, for example, to cancer treatments that target the vascular endothelial growth factor (VEGF), which is a key mediator of tumor growth and angiogenesis. Tumor growth may be suppressed by employing VEGF-blockade therapy. For example, bevacizumab (BV) is a monoclonal anti-VEGF antibody that can be used as a cancer therapy; however, the efficacy of BV varies based on cancer types and between individual patients. By employing the microbubble formulation and ultrasonic techniques described herein, tumor response to BV treatment can be effectively predicted and/or monitored.

The lipid microbubbles described herein have enhanced detectability in vivo for vasculature perfusion and molecular imaging studies. Size-selected microbubbles (e.g., between approximately 4 μm and 10 μm in diameter, in particular, having diameters of 4-5 μm or 6-8 μm) can act as more sensitive contrast agents for vasculature perfusion studies. Moreover, microbubbles having diameters within a range of approximately 4 μm-5 μm and 6 μm-8 μm may produce greater contrast and have longer half-lives in circulation as compared to polydispersed samples that contain mostly microbubbles having diameters within a range of 1 μm-2 μm. In addition, the larger microbubbles enjoy a larger surface area than and thus may have a greater number of labels on the surface thereof. Moreover, the larger diameter microbubbles may take up a greater amount of space in the blood vessels of the tumor vasculature. These features may result in greater adhesion strength to the targets in the vasculature for the larger diameter microbubbles than their smaller diameter counterparts.

Microbubbles can be formulated by emulsifying a lipid formation with a hydrophobic gas, such as sulfur hexafluoride (SF₆) or perfluorobutane (PFB). For example, the lipid formulation for the emulsification can include (in terms of lipid molar ratios) 90% of 1,2-distearoyl-sn-glycero-3-phosphocholine (DSPC), 5% of 1,2-dipalmitoyl-sn-glycero-3-phosphoethanolamine-N-[methoxy(polyethylene glycol)-2000], and 5% of 1,2-distearoyl-sn-glycero-3-phosphoethanolamine-N-[maleimide(polyethylene glycol)-2000] (DSPE-PEG2K-Mal). The maleimide group can serve as a reactive species that binds to sulfhydryl groups, thereby enabling covalent coupling to free thiol groups.

Microbubbles having diameters in the 1 μm to 2 μm range comprise over 90% of freshly generated (or commercially available) lipid-coated microbubbles. However, these smaller microbubbles attenuate strongly without producing much backscatter during ultrasound imaging. These small diameter microbubbles thus act as a negative contrast agent. In contrast, microbubbles having diameters in the 4 μm-5 μm and 6 μm-8 μm size ranges are highly echogenic. In addition, these larger microbubbles exhibit longer contrast persistence. For example, microbubbles having a diameter in the 6 μm-8 μm size range can be circulated for greater than 15 minutes at a dose of 5×10⁸ microbubbles/mL in a 0.1 mL bolus. Thus, the magnitude and duration of ultrasound contrast enhancement can be strongly dependent on the microbubble size distribution.

The total integrated contrast enhancement can be increased significantly (e.g., greater than 10 times) for microbubbles having a diameter of 6-8 μm at a concentration of 5×10⁷ microbubbles/bolus as compared with microbubbles having a diameter of 4-5 μm at the same concentration. Microbubbles having a diameter of 1-2 μm at any concentration may not measurably enhance the integrated ultrasound signal depth and may in fact contribute to signal attenuation. This effect is illustrated in FIG. 1C, where representative time-intensity curves are shown for each size-selected population after a 100 μL bolus injection of 5×10⁷ microbubbles.

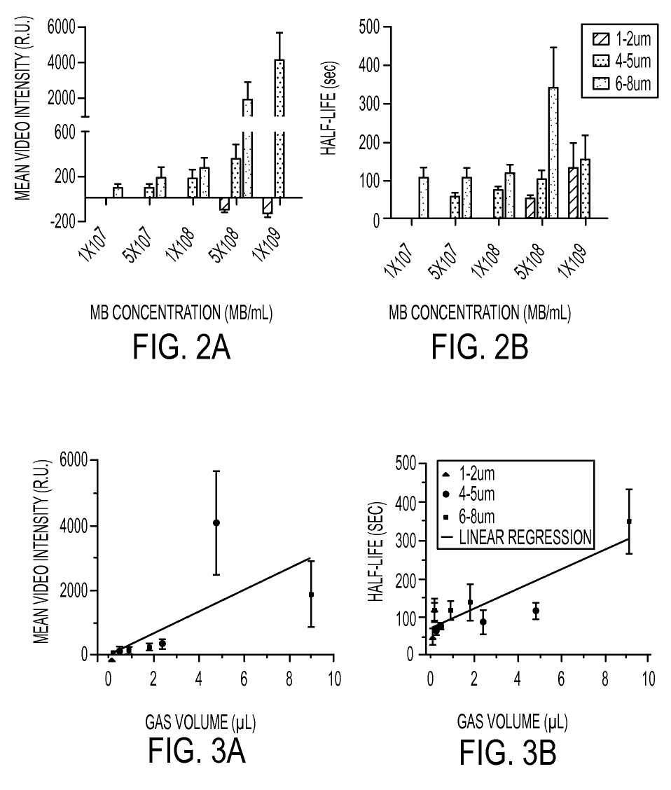

The persistence (i.e., survival in vivo) in circulation can also have important effects on contrast enhancement, molecular imaging, and therapeutic strategies. The duration of the ultrasound contrast signal may be depend on, among other things, the rate of microbubble removal from circulation due to dissolution of the gas core, filtering by the patient's organs, and uptake by macrophages. As shown in FIGS. 2A-2B, for the same concentration, these larger microbubbles may also be more persistent (i.e., survive in vivo) in circulation than smaller microbubbles. However, as reflected in FIGS. 3A-3B, for the same microbubble population gas volume, circulation half-life of the microbubbles may be similar regardless of microbubble diameter.

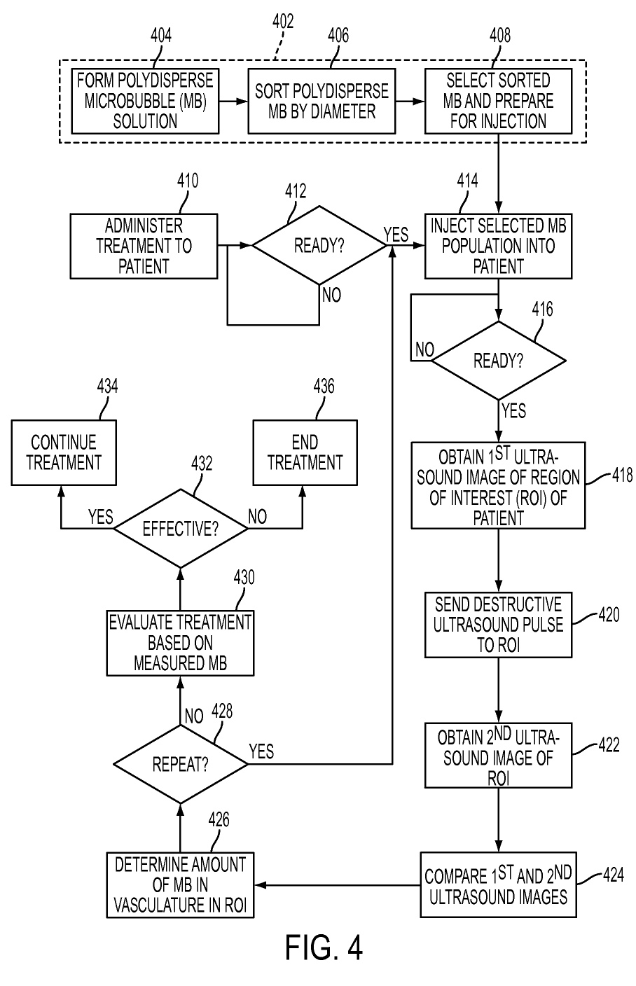

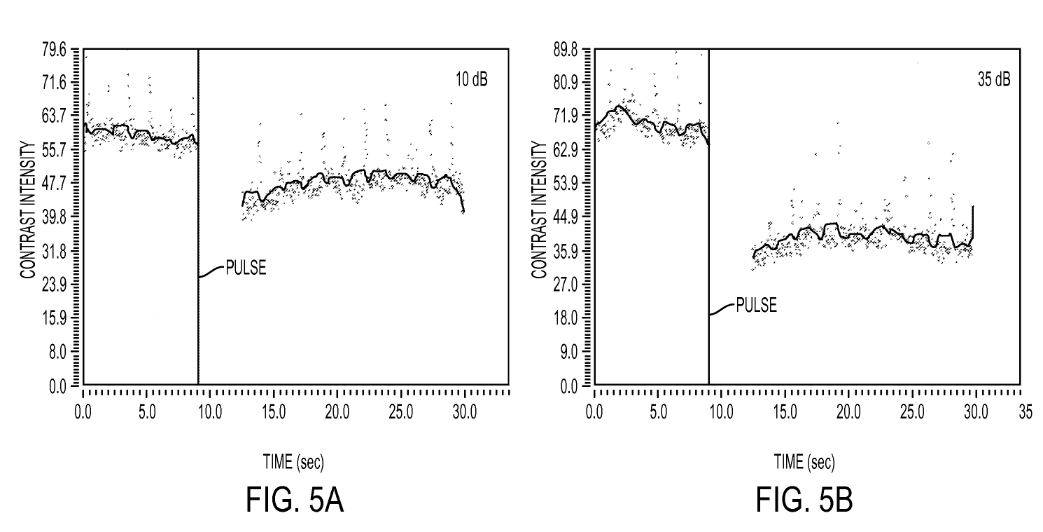

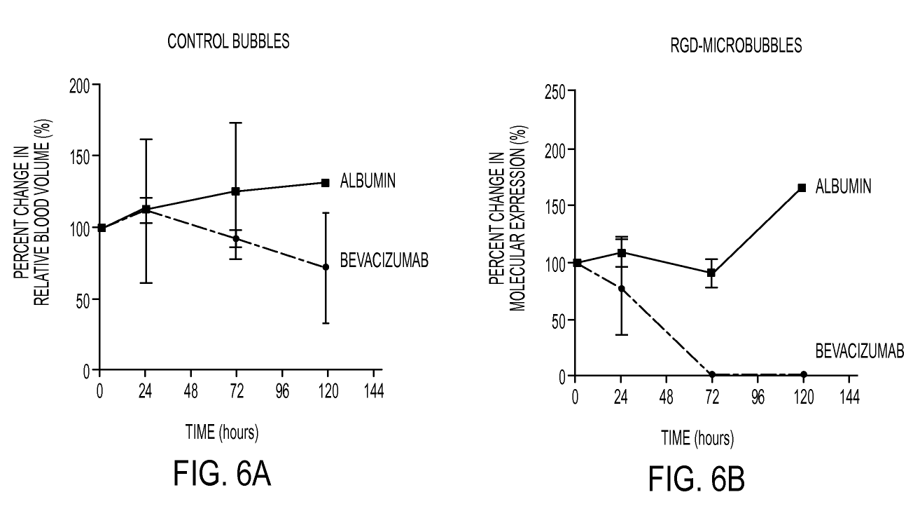

Referring to FIG. 4, a method of determining efficacy of an anti-cancer treatment is shown. In embodiments, a population of microbubbles having diameters within the range from 4 μm to 10 μm can be selected from a polydisperse solution for injecting into a patient at 402. A solution of microbubbles having a variety of diameters therein (e.g., between 0.5 μm and 10 μm) can be first formed at 404. Microbubbles in the polydisperse solution can be size sorted using, for example, differential centrifugation at 406. In such a process, microbubbles can be separated by size based on their relative buoyancy in a centrifugal field. By altering the centrifugal force, viscosity, time, and path length, individual size populations can be separated and collected for use. FIG. 1B shows size distributions of size-selected microbubbles from such a process. The microbubble populations for the individual 1-2 μm, 4-5 μm, and 6-8 μm samples are shown as number-weighted and volume-weighted size distributions. The polydisperse sample shown in FIG. 1B is a microbubble solution that has not undergone differential centrifugation.

A portion of the sorted microbubbles having diameters that result in echogenic microbubbles (i.e., in the range of 4-5 μm and 6-8 μm) can be selected for injection at 408. The resulting size-selected microbubble population can have a mean diameter of, for example, 4.5 μm. Prior to injection, the microbubbles can undergo a post-labeling process to provide peptide molecules on the microbubble surface for binding to target receptors in the desired vasculature. For example, when targeting αᵥβ₃ receptors, cysteine-modified cyclic-RGD peptides can be mixed with the microbubbles, for example, at a ratio of 30:1 (peptide:maleimide molar excess). For a control group (i.e., non-binding), RAD peptides can be mixed with the microbubbles. Incubation may be used to bind the peptides to the maleimide group on the microbubbles. Any unreacted peptides can be removed by appropriate washing. Cysteine can be added and a second incubation performed in order to “cap” any unreacted maleimide groups on the surfaces of the microbubbles.

To determine if a particular treatment administered to a patient at 410 is effective, microbubbles can be injected into the patient after it is determined at 412 that a sufficient time has elapsed after treatment has commenced. Alternatively, injection of microbubbles at 414 can occur just prior to or just after beginning treatment on the patient so as to establish a base line value for the number of microbubbles that bind to receptor sites in the patient's tumor.

At 414, size-selected RGD targeted microbubbles can be injected into the bloodstream of a patient. A tumor within the patient can be simultaneously (or subsequently) monitored by imaging with an ultrasound scanner. At 416, it is determined if the microbubbles have been circulating for a sufficient time to allow the microbubbles to reach and bind to targeted receptor sites in the tumor. For example, the injected microbubbles can be allowed to circulate through the patient for ten minutes before removing any free microbubbles from circulation. To determine the amount of microbubbles that are bound to the αᵥβ₃ integrin receptors, a low frequency destruction pulse can be applied from the ultrasound scanner to destroy any microbubbles that may be within the field of view at 420. Ultrasound images taken before the pulse at 418 and after the destruction pulse at 422 show differences in the image intensity proportional to the amount of targeted bubbles that are present in the vasculature. By comparing the images at 424, a determination can be made regarding the amount of microbubbles present in the tumor vasculature. Control injections using non-binding RAD-peptide bearing microbubbles can be used to correct for RGD-peptide microbubbles that have been passively or non-specifically retained in the tumor vasculature.

The process (e.g., 414-426) can be repeated at 428 at a later time to determine the effect of the treatment on the growth of the tumor. If the treatment is ineffective, the tumor may continue to grow, thereby resulting in an increase in the number of receptor sites in the tumor. As a result, subsequent determinations at 424 at the later time may show an increase in the number of microbubbles present in the tumor vasculature. If the treatment is effective, the tumor may cease growing or may shrink, thereby resulting in the same number of receptor sites or a decrease in the number of receptor sites. As a result, subsequent determinations at 424 at the later time may show the same or decreased number of microbubbles in the tumor vasculature. Based on this evaluation at 430, it can be determined at 432 if the treatment is effective. If effective, the treatment may continue at 434. If ineffective, the treatment may be abandoned at 436 for an alternative treatment. The method of FIG. 4 may also be used with any other treatment to determine efficacy thereof on a cancerous tumor.

To evaluate vascular response (blood volume, perfusion, and molecular expression), two human cancer cell lines (SK-NEP and NGP) were implanted in nude mice and allowed to develop for five weeks. High-frequency ultrasound imaging of the tumors was performed at 40 MHz using a Visualsonics Vevo 770 during bolus tail-vein injections of size-selected microbubble suspensions. The suspensions had a median microbubble diameter of 4.5 μm. The bolus was 504 and the concentration was 5×10⁸ microbubbles/mL. The change in intensity of the ultrasound signal was used to determine the relative blood volume. Targeted microbubbles bearing cyclic RGD-peptides (or cyclic RAD-peptides for the control microbubbles) were used to measure the level of active angiogenesis. The mice were imaged at 0, 1, 3, and 5 days. Immediately after the imaging sessions on days 0 and 3, 0.2 cc of BV (2.5 mg/mL) was administered to the mice. After day 5, the mice were euthanized, and their tumors excised for ex vivo analysis.

In SK-NEP tumor-bearing mice treated with BV, no significant changes in tumor size or relative perfusion was noted. At three days following BV administration, molecular imaging using the RGD-bearing microbubbles showed a significant drop in αᵥβ₃ integrin binding in the tumor region of the treated SK-NEP mice, indicating an interruption in angiogenic activity. In contrast, NGP tumors were unresponsive to BV treatment and continued to increase in size and relative perfusion. No significant differences between BV-treated NGP tumor mice, placebo-treated NGP tumor mice, or placebo-treated SK-NEP mice were observed due to the BV therapy. For tumors that respond to BV treatment, the image intensity of the tumor during treatment may decrease. For example, the difference in image intensity of BV-responsive tumors is substantially reduced (e.g., >95%) after 3 days following treatment. Non-responsive tumors injected with RGD target microbubbles as well as tumors imaged after injection with the control microbubbles were far less affected.

For example, two xenograft model systems were used with previously well-characterized responses to VEGF inhibition, i.e., a responder (SK-NEP-1) and a non-responder (NGP). The two tumor models have divergent responses to VEGF inhibition. Xenografts from the SK-NEP-1 human Ewing family tumor cell line are highly responsive to various anti-VEGF agents, with significant loss of vasculature and inhibition of growth. In contrast, xenografts from the NGP human neuroblastoma cell line continue to grow with only slight restriction and minimally destabilized vessels.

Perfusion-related parameters were examined using the contrast-enhanced ultrasound imaging (CEUS) technique described herein. With CEUS, the high compressibility and resonance behavior of gas-filled microbubbles can make them useful intravascular ultrasound contrast agents, allowing their acoustic signal to be readily differentiated from tissue. CEUS can thus provide real-time imaging at the bedside for qualitative tracking and quantitative measurement of perfusion-related biomarkers. CEUS demonstrated that BV treatment arrested the increase in microbubble perfusion in SK-NEP-1 tumors only. CEUS using RGD-labeled microbubbles, targeting integrin αᵥβ₃ of proliferative vasculature, showed a robust decrease in αᵥβ₃ vasculature following BV treatment in SK-NEP-1 tumors. Response to BV can thus be identified soon after initiation of treatment. The use of this noninvasive ultrasound approach may allow for earlier and more effective determination of efficacy of anti-angiogenic therapy.

The identity of the neuroblastoma cell lines NGP and SK-NEP-1 were verified by STR profiling. The cell lines were stably transfected with FUW-Luciferase plasmid and were selected and maintained in 1 mg/ml neomycin. 1×10⁶ cells were injected intrarenally into 4-6 week old NCR female nude mice. The resulting xenografts were monitored for growth using bioluminescence. At a threshold corresponding to 1-2 g, tumors were randomized to control or treatment groups (cohort size 5-8 mice per modality and treatment groups). BV (0.5 mg) was administered immediately after imaging on days 0 and 3. Animals were killed by CO₂ inhalation at indicated time points (at day 5 after serial imaging studies, and at days 0, 1, 3, and 5 for control and lectin perfusion analyses). At euthanasia, mice were injected with fluorescein-labeled Lycopersicon esculentum lectin (100 μg/100 μl PBS). Vasculature was fixed by infusing 1% paraformaldehyde. 40-μm sections were cut using a vibratome, and digital images subjected to computer-assisted quantitative analysis of tumor vessel architecture.

Size-selected microbubbles described above were used for perfusion and molecular imaging. Lipid-coated, perfluorobutane-filled microbubbles were produced by mechanical agitation. For targeting, a maleimide group was included on the distal end of the polyethylene glycol group on the lipopolymer used to coat the microbubbles. Residual lipid was removed and 4.5-μm median diameter microbubbles were isolated by centrifugation. Targeted microbubbles were then conjugated to cysteine-tagged RGD (target) or RAD (control) peptides and washed again to remove residual peptide. RGD conjugation was confirmed by HPLC and MALDI-TOF. Ultrasound imaging was performed using a small-animal ultrasound scanner with a 30-MHz transducer.

Mice were anesthetized and placed on a physiological monitoring platform, and their tail veins were catheterized for injections using a 27-gauge, ½-inch butterfly catheter. The transducer was positioned at the tumor midsection, and 2-D ultrasound images were acquired using a field of view of 17×17 mm. A 50-μL bolus (2.5×10⁷ microbubbles) followed by a 15-μL saline flush were injected while imaging at the maximum frame rate for respiratory gating (˜11 frames/second) and 100% power. Maximum intensity persistence (MIP) images were acquired using non-targeted RAD-microbubbles. Contrast enhancement was detected using background subtraction from reference videos acquired before the microbubble injection. A time-intensity curve was generated from the MIP image stack by calculating the contrast enhancement within a region of interest (ROI) drawn around the hypoechoic tumor region. Relative microbubble perfusion (rmp) was defined as the maximum signal enhancement and determined by regression of a monoexponential function to the time-intensity curve during microbubble uptake.

For molecular ultrasound imaging, a series of lower frequency pulses was applied to destroy microbubbles in the field of view 10 minutes after microbubble injection. Video images were captured 10 seconds before and after the burst pulse. Contrast before the burst pulse included contributions from both freely circulating and adherent microbubbles, whereas contrast following the burst pulse resulted from only freely circulating microbubbles. The relative targeted microbubble adhesion (rtma) was measured as the difference in the linearized grayscale pixel intensities within the ROI for targeted RGD-microbubbles, minus that for RAD-microbubbles. Use of the RAD-microbubbles provided measurement and subtraction of the signal from microbubbles adherent by nonspecific interactions. RAD- and RGD-microbubble injections were randomized. Thus, the rtma was a measurement of the contrast enhancement within the ROI from only targeted microbubbles, and it did not include contributions from tissue motion, freely circulating microbubbles or nonspecifically adherent microbubbles.

To assess overall differences between the groups of mice, a linear mixed effects regression model was used that estimates linear trajectories for each cohort over time, while accounting for comparisons among repeated measurements from the same mice. The intercept was treated as a random effect and covariate to account for the differences between mice at baseline. The maximum likelihood method was used for estimation of the regression coefficients. The ultrasound perfusion imaging employed a linear model for the raw data at days 0, 1, 3, and 5, using the slope of linear fit for comparison between cohorts. The ultrasound targeted imaging implemented a non-linear model including random effects. The data for each mouse was normalized to the initial value to account for differences in baseline. The data was then fit to an exponential decay (e^{-kt}). The decay constant (k) term was used for comparison between cohorts. The selection of these models and parameters was driven by the different trends observed by the ultrasound imaging modality. Finally, comparisons of individual perfusion-related parameters and lectin perfusion studies between BV- and vehicle-treated and day 0 control tumors at days 1, 3, and 5 were calculated using a two-tailed Student's t-test, with alpha set at 0.05.

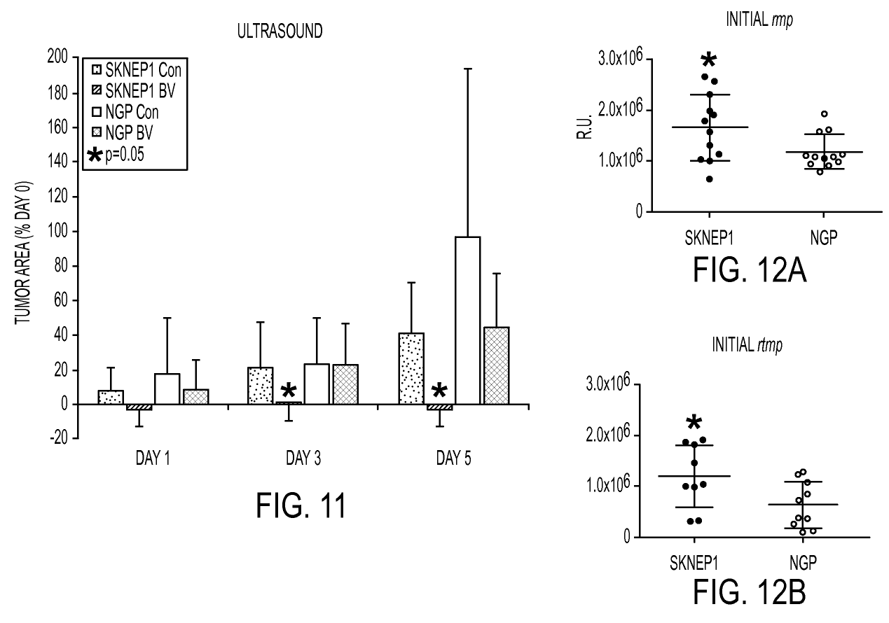

Xenografts formed in the kidney of NCR nude mice with the human Ewing family tumor cell line SK-NEP-1 are highly sensitive to VEGF blockade therapy, while human neuroblastoma NGP cell lines are much less responsive to VEGF blockade therapy. The intrarenally implanted xenograft tumors were monitored for growth and randomly assigned to biweekly injections of anti-VEGF antibody BV or vehicle. Cohorts of tumor-bearing animals were serially imaged at day 0 (pretreatment), and days 1, 3, and 5 after the first drug injection. To confirm the characterization of responsiveness, 2-D tumor area was measured by ultrasonography, the results of which are shown in FIG. 11.

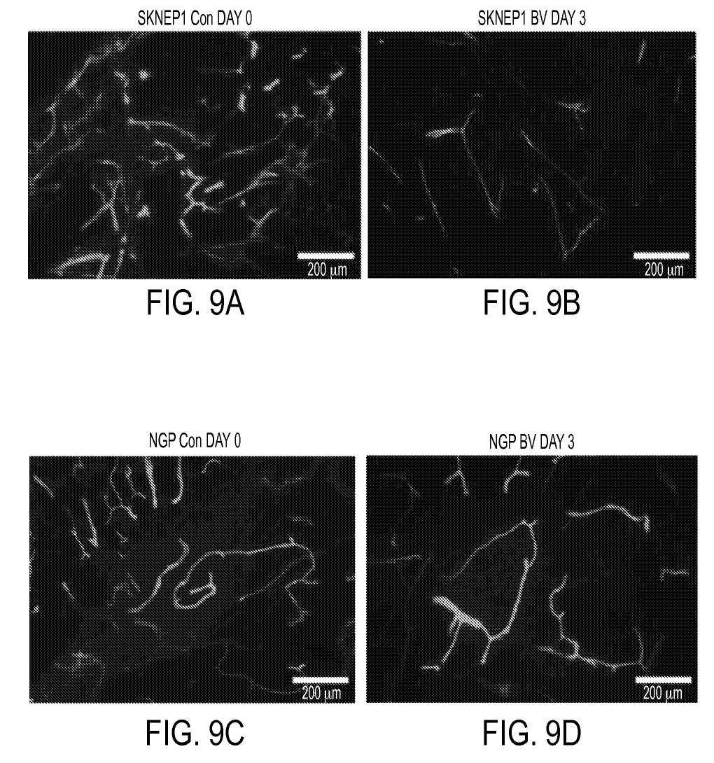

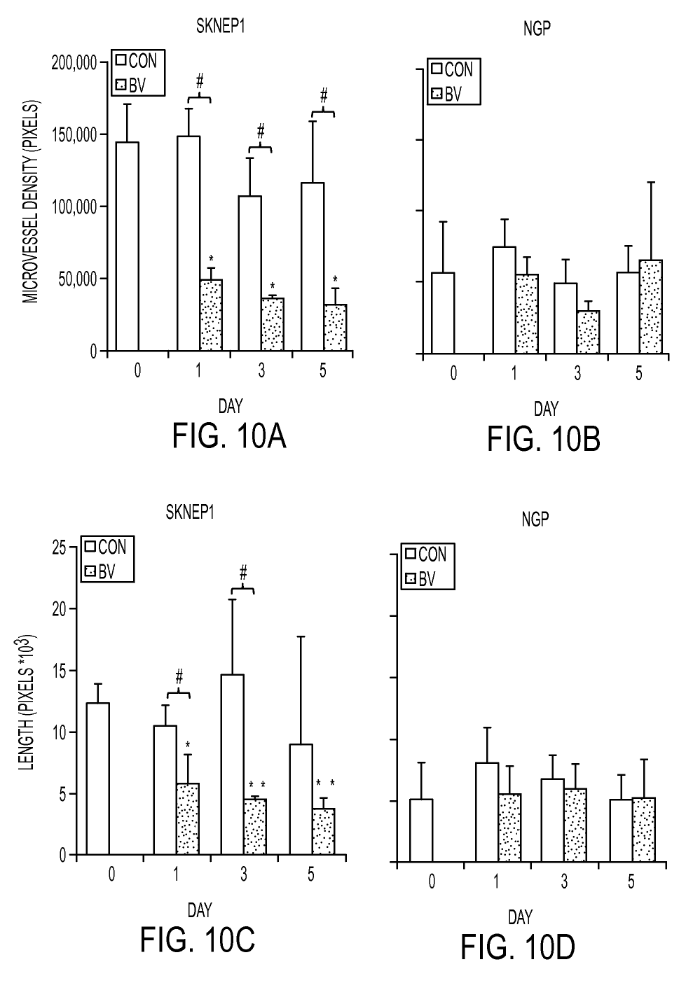

The tumor 2-D cross-sectional area was determined from the ultrasound images using Visualsonics software. Area measurements of the tumor were calculated from the ultrasound images using a ROI that encompassed the hypoechoic region of the kidney (tumor tissue). All area measurements were performed in the midsection of the tumor. As reflected in FIG. 11, BV treatment significantly arrested tumor growth in SK-NEP-1 xenografts at days 3 and 5 in comparison to control, but not in NGP xenografts, Thus, treatment of SK-NEP-1 mice with BV essentially arrested tumor growth over the 5 day period (i.e., ultrasound −5%), as compared with continued growth in the control tumors (i.e., ultrasound +42%). Growth of NGP tumors was unaffected by BV treatment (i.e., ultrasound +44% vs. +97%, BV vs. control). These results along with analysis of lectin perfusion studies of the vasculature (see FIGS. 9A-9D and 10A-10D), verify the classification of SK-NEP-1 as a responder and NGP as a non-responder to VEGF blockade therapy.

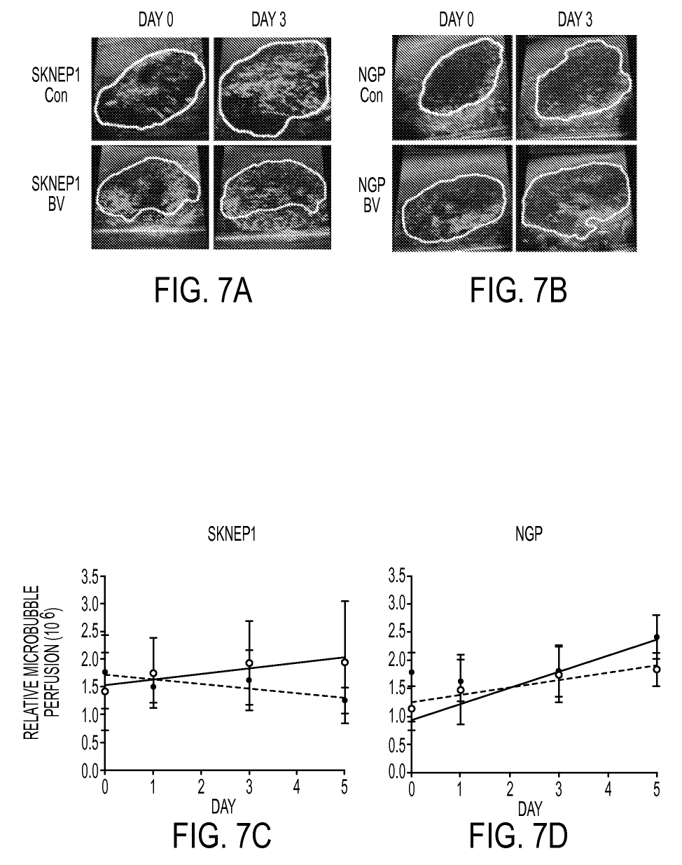

Microbubble contrast agents exhibit hemodynamics similar to erythrocytes, allowing measurement of blood flow using ultrasound (see FIGS. 7A-7B). Size-selected microbubbles optimized for perfusion imaging were used. NCR Nude mice implanted with either NGP or SK-NEP-1 renal tumors were imaged with high-frequency ultrasound following bolus injections of size-selected microbubbles. Mice were imaged at 0, 1, 3, and 5 days. Bevacizumab (BV) or albumin (Con) were administered immediately after the imaging sessions on days 0 and 3. Representative tumors with microbubble perfusion overlays from the SK-NEP-1 and NGP groups are shown at day 0 and day 3. Hypoechoic tumor regions are outlined in white and regions of microbubble perfusion are colored green (i.e., spots within the outlined region in the figures). In contrast to the SK-NEP-1 Con, NGP Con, and NGP BV groups, the BV-treated SK-NEP-1 tumors showed no increase in size or microbubble perfusion.

Relative microbubble perfusion (rmp) was measured for each animal prior to treatment. The average rmp was approximately 40% higher for SK-NEP-1 tumors compared to NGP (P=0.035) (see FIG. 11), indicating that the initial perfusion was higher in SK-NEP-1 then in NGP xenografts. Looking at response to therapy, the mean rmp in BV-treated SK-NEP-1 tumors remained unchanged 1, 3 and 5 days after treatment (P=0.67, 0.66 and 0.40, respectively) (see FIGS. 7C-7D). Linear regression lines were applied to the mean rmp values from 0 to 5 days for the Con (---) and BV ( [symbol] ) groups. The slope of the regression lines for Con (empty circle) and BV-treated (filled circle) tumors were compared for the SK-NEP-1 (n=7 and 6, respectively) and NGP cohorts (n=6 and 7, respectively) using a linear mixed-effects model.

BV-treated NGP tumors, on the other hand, showed an increase in mean rmp of 38±21% (P=0.0004) by day 1, 57±48% (P=0.0056) by day 3 and 105±41% (P<0.00001) by day 5. For the control groups, mean rmp increased for both SK-NEP-1 and NGP tumors by day 5 (P=0.034 and 0.0096, respectively). As above, a linear mixed-effects model was used to evaluate the combined effects over 5 days (see FIGS. 7C-7D). The slopes of the linear regression between BV-treated and control SK-NEP-1 cohorts were statistically different (P=0.0044), while again no difference between the NGP treated and control group was observed (P=0.25). Thus, CEUS perfusion imaging showed that BV treatment arrested the increase in microbubble perfusion in responder SK-NEP-1 tumors, but not in NGP xenografts.

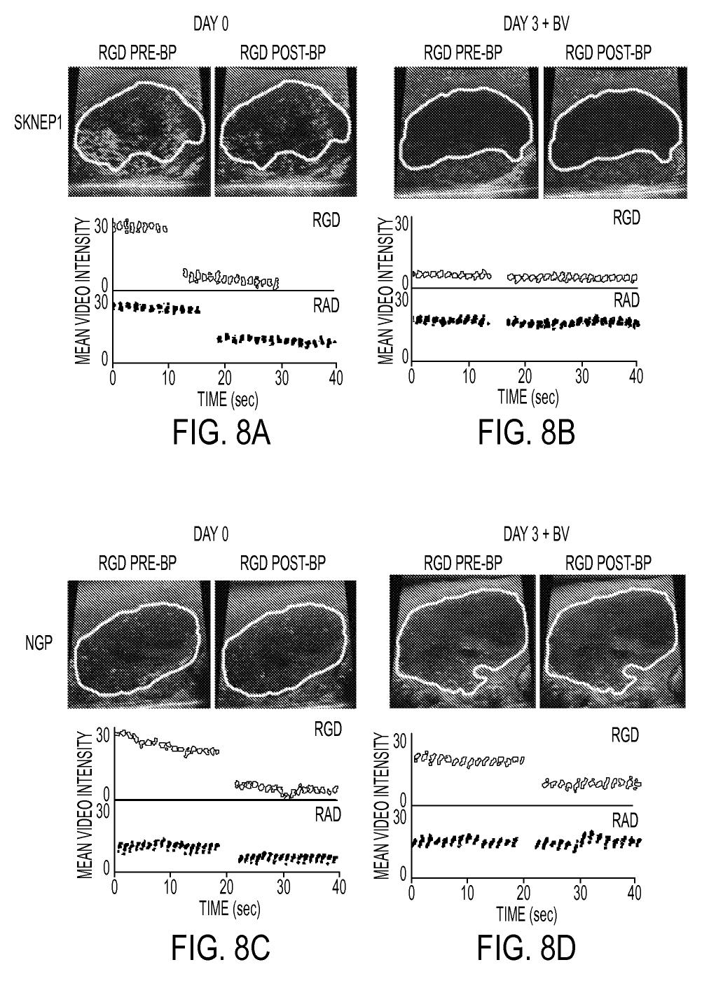

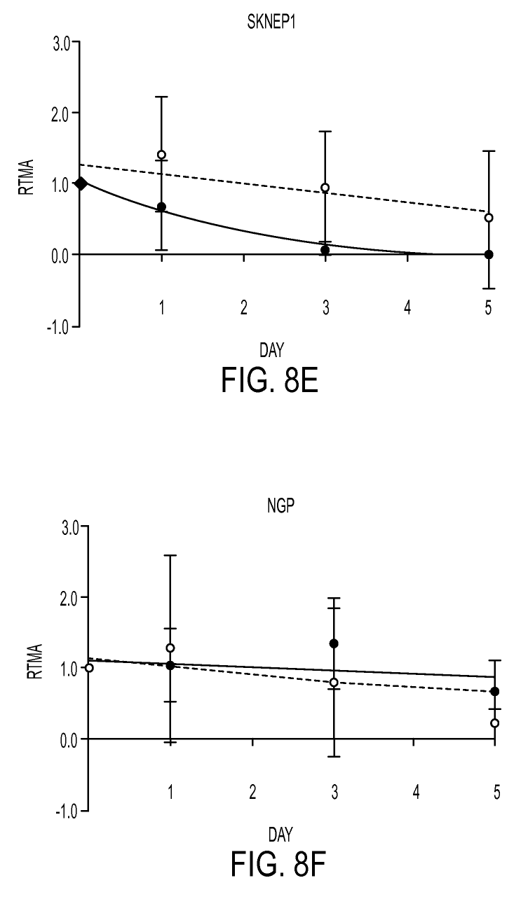

CEUS was also used to monitor αᵥβ₃ integrin, which is expressed preferentially on actively proliferating vessels found in growing tumors. Microbubbles were targeted to this epitope by surface conjugation of RGD peptide (RAD peptide serving as control). Relative targeted microbubble adhesion (rtma) was quantified by the decrease in tumor pixel intensity following the ultrasound microbubble-burst pulse (versus RAD control) (see FIGS. 8A-8F). The expression of αᵥβ₃ integrin in the vessels of the tumor region was evaluated in the same mice using CEUS with RGD-labeled microbubbles (versus RAD control). Representative tumors with microbubble contrast overlays from the SK-NEP-1 and NGP groups are shown at day 0 and day 3 following a 10-min dwell time after the bolus injection (5×10⁸ mL⁻¹, 50 μL). Images are shown before (left) and after (right) the burst pulse was applied to fragment the microbubbles in the field of view for FIGS. 8A-8D. The corresponding video intensity-time curve is shown below each pair of images for both RGD-labeled (top trace) and RAD-labeled (bottom trace) microbubble injections.

Before treatment, mean rtma for SK-NEP-1 was approximately 35% higher than that for NGP (P=0.038) (See FIG. 12B). Investigating the response to therapy, BV-treated SK-NEP-1 mean rtma did not change significantly after 1 day, but it decreased 91±5% (P<0.00001) by day 3 and 99±5% (P<0.00001) by day 5. The mean rtma values decreased also for BV-treated NGP and control mice, but at slower rates. The decay rates were compared using a non-linear exponential decay model (see FIGS. 8E-8F). The rtma was quantified in the tumor region on days 0, 1, 3, and 5. The data was normalized to the baseline value for each mouse to correct for differences in the initial values. An exponential curve fit (e^{-kt}) was applied to the Con (---) and BV ( [symbol] ) groups using a non-linear mixed-effects model. The decay constant (k) for Con (empty circle) and BV-treated (filled circle) groups were compared for the SK-NEP-1 (n=7 and 6, respectively) and NGP cohorts (n=7 and 7, respectively). The difference in decay rates between BV-treated and control SK-NEP-1 were statistically different (P=0.022), while no difference was observed between treated and control NGP (P=0.26). Thus, ultrasound molecular imaging showed a robust decrease in αᵥβ₃ integrin expression as early as 3 days following BV treatment only for the responder, SK-NEP-1, and not the other groups.

Quantified changes in lectin perfusion studies of tumor vasculature were consistent with changes detected by CEUS. Established SK-NEP-1 and NGP tumors were injected IV with fluorescein-labeled L. esculentum lectin, prior to sacrifice at day 0, or after 1, 3, or 5 days of treatment with either vehicle (Con) or bevacizumab (BV). FIGS. 9A-9D are representative fluorescent images at days 0 and 3 of BV treatment. After binarization of the images, microvessel density (MVD) was estimated by the total number of white pixels per field. The results shown in FIGS. 10A-10B are as the mean pixel count per image +SD. BV significantly decreased MVD in SK-NEP-1 at Days 1, 3, 5 (*p<0.003), but not in NGP.

As compared to day 0 controls, MVD in BV-treated SK-NEP-1 tumors decreased by 66% at day 1, 75% at day 3, and 78% at day 5 (P=0.003, each), whereas MVD did not change in BV-treated NGP tumors (see FIGS. 9A-9D and 10A-10B). Control SK-NEP-1 and NGP tumor perfusion also did not change over the experimental period.

Computer-assisted image analysis was used to examine changes in specific vessel features. If vascular area is calculated from total pixels, large-diameter vessels contributed disproportionately to MVD as compared to fine capillaries. To more closely estimate numbers of vessels, total length can be calculated by skeletonizing images and then scoring these by computer. BV treatment significantly decreased total vessel length in SK-NEP-1 at days 1, 3, 5 (P=0.01, P=0.01, P=0.001), but not in NGP tumors (as shown in FIGS. 10C-10D). New vascular branches can form a dynamic and relatively VEGF-dependent element in angiogenic networks. Similar to the pattern of change detected in the αᵥβ₃ integrin-expressing vessels using RGD-tagged microbubbles, bevacizumab significantly decreased total vascular branch number in SK-NEP-1 at days 3 and 5 (P=0.014), but not in NGP (data not shown).

BV treatment thus reduced overall perfusion in SK-NEP-1 tumors, with disproportionate pruning of smaller, branch vessels. In particular, BV decreases microvessel density (MVD), vessel length, and total vessel number in SK-NEP-1 tumors, but not NGP tumors. Prognostic imaging biomarkers (mean relative microbubble perfusion (proportional to total blood flow) and relative targeted microbubble adhesion (proportional to αᵥβ₃ integrin concentration on the luminal surface of the endothelium)) related to blood perfusion can be identified using CEUS imaging. Using the disclosed techniques, assessment of tumor response in patients (e.g., humans or other animal) can be obtained at an early stage of therapy, with the potential to guide physicians in optimizing treatment. Moreover, ultrasound is well-suited for real-time assessment in animals and humans, both children and adults, with inexpensive, widely available, and portable equipment, and rapid imaging times.

The use of the disclosed microbubbles targeted to cancer endothelial biomarkers may allow CEUS imaging to provide a first-line modality for diagnosing and monitoring cancer angiogenesis. For patients with non-responsive tumors, alternate regimens could be considered without waiting for overt therapeutic failure to occur, avoiding needless toxicity. Alternatively, those patients whose tumors demonstrated responsiveness could remain on treatment. Lastly, given the high cost of biologically-targeted therapies like BV, such early assessment of drug effectiveness could reduce the economic strains of cancer treatment for patients and families.

Although the description above pertains to the monitoring of the efficacy of BV treatment of cancer, the methods, systems, and devices of the present disclosure are applicable to other treatments as well. In addition, the microbubbles can be used in the imaging of other types of tumors beyond those described herein. By appropriate selection of microbubble surface chemistry, the microbubbles can be used to monitor the efficacy of a variety of conditions, treatments, and diseases, as well as different types of cancer/tumors. Moreover, embodiments and teachings of the present disclosure are applicable to more than just monitoring treatment efficacy. Rather, the teachings and embodiments of the disclosed subject matter may be applied to health monitoring of patient vasculature or any other in vivo vasculature inspection application, according to one or more contemplated embodiments.

Furthermore, the foregoing descriptions apply, in some cases, to examples generated in a laboratory, but these examples can be extended to production techniques. For example, where quantities and techniques apply to the laboratory examples, they should not be understood as limiting. In addition, although specific chemicals and materials have been disclosed herein, other chemicals and materials may also be employed according to one or more contemplated embodiments. For example, although the production of microbubbles with a hydrophobic gas has been specifically described herein, other gases (elemental or compositions) are also possible according to one or more contemplated embodiments

Features of the disclosed embodiments may be combined, rearranged, omitted, etc., within the scope of the invention to produce additional embodiments. Furthermore, certain features may sometimes be used to advantage without a corresponding use of other features.

It is, thus, apparent that there is provided, in accordance with the present disclosure, specially formulated microbubbles, methods for producing said microbubbles, and systems, methods, and devices for ultrasonic assessment of cancer and response to therapy. Many alternatives, modifications, and variations are enabled by the present disclosure. While specific embodiments have been shown and described in detail to illustrate the application of the principles of the invention, it will be understood that the invention may be embodied otherwise without departing from such principles. Accordingly, Applicants intend to embrace all such alternatives, modifications, equivalents, and variations that are within the spirit and scope of the present invention.

1. A method for determining efficacy of treatment of a cancerous tumor in a patient, the method comprising:
at a first time after administering the treatment to a patient, injecting a population of microbubbles into the patient, the population of microbubbles being size-selected so as to have diameters of 4-5 μm or 6-8 μm, each microbubble having a surface chemistry that targets receptor sites in said tumor;
after the injecting, imaging a field of view using ultrasound so as to obtain a first image, the field of view including at least a portion of said tumor;
after the imaging, sending an ultrasonic pulse to said field of view so as to destroy the microbubbles in said field of view, the ultrasonic pulse having a higher intensity than the ultrasound waves used for said imaging;
re-imaging the field of view using ultrasound so as to obtain a second image; and
comparing the intensity of the first and second images so as to measure the number of microbubbles attached to the targeted receptor sites in said tumor.

2. The method of claim 1, further comprising:
repeating the injecting, imaging, sending, re-imaging, and comparing steps at a second later time after the administering the treatment to a patient; and
determining the efficacy of the treatment based on the measured number of microbubbles at the first time and at the second later time.

3. The method of claim 2, wherein said treatment is determined to be effective when the measured number of microbubbles at the second later time is substantially less than the measured number of microbubbles at the first time.

4. The method of claim 2, wherein the second later time is at least three days after administering the treatment to the patient.

5. The method of claim 4, wherein said treatment is determined to be effective when the measured number of microbubbles at the second later time is reduced by 95% as compared to the measured number of microbubbles at the first time.

6. The method of claim 2, further comprising discontinuing said treatment if the measured number of microbubbles at the second later time is substantially the same or greater than the measured number of microbubbles at the first time.

7. The method of claim 1, further comprising administering the treatment to the patient.

8. The method of claim 1, further comprising:
producing a solution of microbubbles by mechanically agitating a lipid solution in the presence of a hydrophobic gas;
isolating microbubbles having diameters of 4-5 μm and 6-8 μm from the solution using centrifugation; and
post-labeling the isolated microbubbles with peptides to form said population of microbubbles for injection.

9. The method of claim 8, wherein said post-labeling includes:
binding the peptides to maleimide groups on each microbubble surface; and
capping unreacted maleimide groups on each microbubble surface after said binding.

10. The method of claim 8, wherein said lipid solution includes 90% of 1,2-distearoyl-sn-glycero-3-phosphocholine (DSPC), 5% of 1,2-dipalmitoyl-sn-glycero-3-phosphoethanolamine-N-[methoxy(polyethylene glycol)-2000], and 5% of 1,2-distearoyl-sn-glycero-3-phosphoethanolamine-N-[maleimide(polyethylene glycol)-2000] (DSPE-PEG2K-Mal).

11. The method of claim 1, wherein the surface chemistry includes RGD peptides arranged on the microbubble surface so as to bind to αᵥβ₃ integrin receptors in said tumor.

12. The method of claim 1, wherein said treatment includes bevacizumab.

13. A substance for investigation of the efficacy of an anti-cancer treatment comprising:
a plurality of microbubbles in solution, each microbubble having a gas core surrounded by a lipid membrane, the lipid membrane having a surface chemistry that binds to receptor sites in a cancerous tumor,
wherein the respective diameters of the plurality of microbubbles is within a range of 4-5 μm or 6-8 μm.

14. The substance of claim 13, wherein said surface chemistry includes maleimide groups on each microbubble surface.

15. The substance of claim 14, wherein said surface chemistry includes a peptide bound to one of the maleimide groups.

16. The substance of claim 15, wherein another of the maleimide groups is capped with cysteine.

17. The substance of claim 13, wherein the targeted receptor sites include αᵥβ₃ integrin receptors.

18. The substance of claim 13, wherein the surface chemistry includes a ligand that binds to the targeted receptor sites.

19. The substance of claim 13, wherein the surface chemistry includes RGD peptides.

20. The substance of claim 13, wherein the gas in said core is a hydrophobic gas.

21. The substance of claim 20, wherein said hydrophobic gas is one of SF₆ or PFB.

22. The substance of claim 13, wherein the surface chemistry is such that the microbubble can bind to one of the targeted receptor sites in the cancerous tumor.

23. The substance of claim 13, wherein the lipid membrane is formed from an emulsification including 90% of 1,2-distearoyl-sn-glycero-3-phosphocholine (DSPC), 5% of 1,2-dipalmitoyl-sn-glycero-3-phosphoethanolamine-N-[methoxy(polyethylene glycol)-2000], and 5% of 1,2-distearoyl-sn-glycero-3-phosphoethanolamine-N-[maleimide(polyethylene glycol)-2000] (DSPE-PEG2K-Mal).
